(19) United States
(12) Patent Application Publication
(10) Pub. No.: US 2021/0313064 A1
(43) Pub. Date: Oct. 7, 2021

(54) TAU PROTEIN ACCUMULATION PREDICTION APPARATUS USING MACHINE LEARNING AND TAU PROTEIN ACCUMULATION PREDICTION METHOD USING THE SAME

(71) Applicant: SAMSUNG LIFE PUBLIC WELFARE FOUNDATION, Seoul (KR)

(72) Inventors: Sang Won SEO, Seoul (KR); Hye Joo LEE, Seoul (KR); Jae Ho KIM, Sejong-si (KR)

(21) Appl. No.: 17/211,003

(22) Filed: Mar. 24, 2021

(30) Foreign Application Priority Data
Mar. 30, 2020 (KR) 10-2020-0038627

(51) Int. Cl. G16H 50/20; G16B 40/00; G06N 20/20

(52) U.S. Cl. CPC G16H 50/20; G06N 20/20; G16B 40/00

(57) ABSTRACT

Disclosed is herein a tau protein accumulation prediction method that includes: a process of inputting: at least one of neuropsychological test information, APOE4 genotype information, positron emission tomography (PET) information, atrophy information of a hippocampal volume, and atrophy information of a cerebral cortical thickness; clinical information; and mild cognitive impairment expression stage information; and a process of calculating a prediction result indicating whether or not a tau protein is accumulated on the brain. According to the tau protein accumulation prediction method, severity or prognosis of a brain disease can be predicted.

### BACKGROUND

### 1. Technical Field

The present disclosure relates to a method and apparatus for predicting the prognosis of a brain disease using a tau protein and, more particularly, to a method and apparatus for predicting the prognosis of a brain disease by predicting whether or not a tau protein will be accumulated on a brain, using machine learning.

### 2. Related Art

Recently, with the aging progress, a population having an impairment of a cognitive function due to the Alzheimer's disease (AD) has increased. Most cures for the AD developed so far are mostly cholinesterase inhibitors (ChEIs), and the remaining cures are N-methyl-D-aspartate (NMDA) receptor antagonists. These cures have a limitation that medical benefits thereof are exhibited in an initial stage of a disease.

Meanwhile, proteins known as a cause of the AD include a tau protein, amyloid-β, and so on. A progress degree of the AD can be determined according to whether or not these causal proteins are accumulated on the brain and a decline in the cognitive function can be predicted. Therefore, these causal proteins can serve as biomarkers of the AD.

To reduce a curative speed or a brain disease progress speed in the initial stage of the brain disease, there is a need for an initial diagnosis of mild cognitive impairment (MCI) and the AD, and importance of discrimination of these causal proteins as the biomarkers is increased.

For the purpose of the discrimination of the tau protein among them, resultant information obtained by using various tools such as a neuropsychological factor, a genetic factor, an aged population statistical factor, and brain image based tools such as magnetic resonance imaging (MRI), computer tomography (CT), positron emission tomography (PET), and single photon emission computed tomography (SPECT) for evaluating the cognitive function may be used as a variable of analysis for the discrimination of the tau protein, and a discrimination tool of the tau protein is gradually diversified.

In connection with this, a method of precluding levels of a tau protein and an amyloid-β peptide, and an AD in a cerebrospinal fluid (CSF) or determining them as secondary danger factors using a brain image-based tool (PET, SPECT, or MRI) is disclosed in Korean Unexamined Patent Application Publication No. 10-2016-0116351 (POLYUNSATURATED FATTY ACIDS FOR TREATMENT OF DEMENTIA AND PRE-DEMENTIA-RELATED CONDITIONS).

However, because a type of information generated from existing diversified tools for the discrimination of the tau protein has a restrictive critical point, and validity intended for each piece of information is not evaluated, prediction results of new information that synthetically considers each piece of information do not have reliability.

Thus, there is a need to utilize machine learning in order to combine the pieces of information generated from the diversified discrimination tools and in order to derive a result having reliability.

### PRIOR ART DOCUMENT

### Patent Document

(Patent Document) Korean Unexamined Patent Application Publication No. 10-2016-0116351

### SUMMARY

Various embodiments are directed to providing a tau protein accumulation prediction apparatus and method capable of comparing and analyzing resultant information produced from diversified tau protein discrimination tools through the tau protein accumulation prediction apparatus, and making a combination of diversified tools suitable for a subject.

Further, various embodiments are directed to providing an apparatus and method capable of comparing and analyzing reliability of tau protein discrimination tools.

Further, various embodiments are directed to providing a tau protein accumulation prediction apparatus using machine learning capable of providing a new algorithm for predicting a load of a tau protein accumulated on the brain of a prodromal Alzheimer's disease subject using clinical information, neuropsychological test resultant information, and brain structural change information of the subject.

In an embodiment, a tau protein accumulation prediction method according to the present disclosure may include: a process of inputting: at least one of neuropsychological test information, APOE4 genotype information, positron emission tomography (PET) information, atrophy information of a hippocampal volume, and atrophy information of a cerebral cortical thickness; clinical information; and mild cognitive impairment expression stage information; and a process of calculating a prediction result indicating whether or not a tau protein is accumulated on the brain.

In some embodiments, the process of calculating a prediction result may include a process of analyzing whether or not a tau protein load is accumulated, using a machine learning algorithm out of classification analysis models.

In some embodiments, the machine learning algorithm may include a tree-based model.

In some embodiments, the tree-based model may include one of a gradient boosting machine (GBM) model and a random forest (RF) model.

In some embodiments, the clinical information may include at least one of an age, a gender, and educated years of a subject.

In another embodiment, a tau protein accumulation prediction apparatus according to the present disclosure may include: an input unit configured to receive: at least one of neuropsychological test information, APOE4 genotype information, positron emission tomography (PET) information, atrophy information of a hippocampal volume, and atrophy information of a cerebral cortical thickness; clinical information; and mild cognitive impairment expression stage information; and a processor configured to calculate a prediction result indicating whether or not a tau protein is accumulated on the brain.

In some embodiments, the processor may analyze whether or not a tau protein load is accumulated, using a machine learning algorithm out of classification analysis models.

In some embodiments, the machine learning algorithm may include a tree-based model.

In some embodiments, the tree-based model may include one of a gradient boosting machine (GBM) model and a random forest (RF) model.

In some embodiments, the clinical information may include at least one of an age, a gender, and educated years of a subject.

The tau protein accumulation prediction apparatus using machine learning according to the present disclosure having the above-described configuration has considerable accuracy in predicting a load of a tau protein accumulated on the brain.

Further, the tau protein accumulation prediction apparatus using machine learning according to the present disclosure can provide information for sorting a subject group intended for the tau protein.

In addition, the tau protein accumulation prediction apparatus using machine learning according to the present disclosure can predict the severity or prognosis of a disease.

### BRIEF DESCRIPTION OF THE DRAWINGS

FIG. 1 is a flow chart illustrating an included or excluded subject among data sets of ADNI.

FIG. 2A illustrates an ROC curve of a GBM model, and FIG. 2B illustrates an ROC curve of an RF model.

FIG. 3A illustrates importance of factors having an influence on accumulation of a tau protein in a GBM model, and FIG. 3B illustrates importance of factors having an influence on accumulation of a tau protein in an RF model.

FIG. 4 illustrates demographic information and a clinical characteristic.

FIG. 5 illustrates a machine learning-based prediction model having a different combination of biomarkers.

FIG. 6 illustrates relative function importance of a GBM model and an RF model.

### DETAILED DESCRIPTION

Hereinafter, the terms used herein will be described in brief, and configurations and operations of exemplary embodiments of the present disclosure will be specifically described as specific contents for carrying out the present disclosure.

Terms used in the present disclosure adopt general terms that are currently widely used as possible by considering functions in the present disclosure, but the terms may be changed depending on an intention of those skilled in the art, precedents, and emergence of new technology. Further, in a specific case, a term which an applicant arbitrarily selects is present, and in this case, a meaning of the term will be disclosed in detail in a corresponding description portion of the disclosure. Accordingly, the terms used in the present disclosure should be defined based on not just names of the terms but meanings of the terms and contents throughout the present disclosure.

In the entire specification, when a certain portion “includes” a certain component, this indicates that the other components are not excluded, but may be further included unless specially described to the contrary. The terms “unit”, “module”, etc. described in the specification indicate a unit for processing at leapt one function or operation, which may be implemented by hardware, software or a combination thereof. Further, in the entire specification, when an element is referred to as being “connected” or “coupled” to another element, this includes a case where it can be “directly connected or coupled” to the other element or a case where it can be connected to the other element “via an intervening element”.

Hereinafter, embodiments of the present disclosure will be described in detail with reference to the attached drawings such that those having ordinary knowledge in the art to which the present disclosure pertains can easily carry out the embodiments. However, the present disclosure may be implemented in several different forms, and is not limited to the embodiments described here. Portions irrelevant to the description are omitted in the drawings in order to clearly describe the present disclosure, and similar portions throughout the specification are given similar reference signs.

Whether or not a tau protein is accumulated on the brain may be an important marker that predicts a cognitive function and a cognitive decline. A load of the tau protein accumulated on the brain in a prodromal Alzheimer's disease (AD) may be exhibited in various forms.

Meanwhile, to apply a positron emission tomography (PET) image for detecting the load of the tau protein accumulated on the brain, considerable expenses are required, and facilities having PET equipment are limited.

Accordingly, a prediction model for detecting the prodromal AD prior to photographing the PET image of the tau protein accumulated on the brain is required, and the present disclosure relates to a tau protein accumulation prediction apparatus using a machine learning algorithm in the prodromal AD.

The prodromal AD may indicate mild cognitive impairment (MCI) before dementia. Meanwhile, a progress process of pathophysiological dementia may be divided into a preclinical AD, MCI resulting from the AD, a prodromal AD, and AD dementia.

The tau protein may serve to maintain a discharge passage of, for instance, internal waste materials of nerve cells. Meanwhile, in the case where phosphate more than necessary is coupled to the tau protein, a fibering phenomenon of a protein occurs and tangles. Thereby, an ability to discharge the waste materials of the nerve cells is lowered, and the nerve cell itself becomes a waste material. A poisonous substance is accumulated, and a nerve tissue dies. The AD may make progress due to the death.

Hereinafter, the present disclosure will be described in more detail through embodiments. These embodiments are only for describing the present disclosure in more detail, and it will be apparent to those having ordinary skill in the art that the scope of the present disclosure according to the subject matter of the present disclosure is not limited by these embodiments.

### Embodiment 1: Data Gaining Process

According to an embodiment, data sets of Alzheimer's disease neuroimaging initiative (ADNI) consisting of subjects of ADNI may be used. ADNI is a research project that is initiated as a private-public partnership in 2003.

According to an embodiment, a group of amyloid-β positive MCI subjects may be selected using an ADNI-3 data set.

According to an embodiment, subjects are aged from 55 to 90, are educated over 6 years, are fluent in Spanish or English, and may not have other important neurological diseases.

According to an embodiment, MCI subjects may have a subjective memory complaint whose clinical dementia rating (CDR) point is 0.5 (Petersen et al., 2010). MCI (early and late) subjects correspond to a Logical Memory stage II of the Wechsler Memory Scale (WMS) (early MCI subjects have education adjustment points between about 0.5 and 1.5 SD compared to a normal cognitive average).

An abnormal tau subject in a tree-based model may be defined as a case over a Braak stage III/IV (Braak et al., Neurobiol. Aging, 2003, 24, 197-211). In other words, in the case where a Braak stage in vivo is over III/IV through the tree-based model, a subject may be defined as having an abnormal tau (T+). The AD and a Parkinson's disease may be neuropathologically classified into six stages by the Braak stage.

According to an embodiment, the tree-based model may be a conditional inference tree approach. The conditional inference tree approach is a statistics-based method using a non-parameter test as a correction division criterion with respect to various tests in order to avoid overfitting, and does not require a prejudice prediction selection result and pruning. The conditional inference tree approach includes a decision tree-structured regression model, and determines the Braak stage in vivo based on AV1451 absorption.

According to an embodiment, the conditional inference tree approach may classify all the subjects into a Braak stage V/VI, Braak stage III/IV, Braak stage I/II, or Braak stage 0 group.

According to an embodiment, MCI subjects who undergo 3.0T MRI scanning, florbetapir (¹⁸F)-AV45 PET, and flortaucipir (AV1451) PET in a usual state may be included.

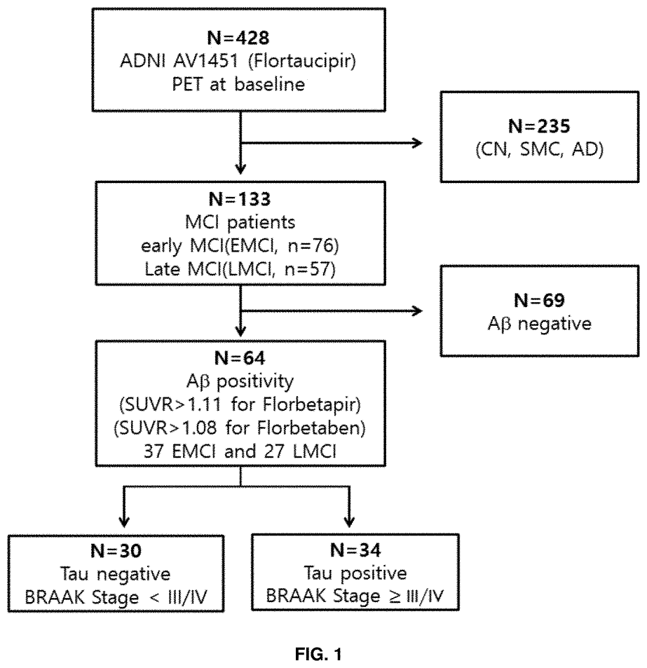

FIG. 1 is a flow chart illustrating an included or excluded subject out of data sets of ADNI.

Referring to FIG. 1, the total number of the data sets of ADNI is 428, which may be calculated in consideration of AV1451 PET results in a usual state. The present disclosure is intended for a patient in a prodromal AD state or an MCI state, and 235 data sets of cognitive normal (CN) patients, AD diagnosis confirmed patients, and significant memory concern (SMC) patients are preferably excluded from 428 data sets. 69 data sets of amyloid-β negative subjects are excluded from 133 data sets after the subtraction. 64 data sets may be divided into 34 data sets over Braak stage III/IV and 30 data sets below Braak stage III/IV.

### Embodiment 1-1: Cortical Thickness Measurement

First, to measure a local cortical thickness of each subject, all T1 volumes may be scanned for structure image analysis through a CIVET pipe line. In other words, a basic MRI image may be registered with MNI-152 template using linear transformation. According to an embodiment, N3 algorithm may be used to correct an image for intensity non-uniformity caused by non-uniformity of a magnetic field.

Next, tissue classification may be performed with a white matter (WM), a gray matter (GM), a cerebrospinal fluid (CSF), and a background (BG) on the basis of a T1 volume scan image. Inner and outer surfaces of the cortex may be automatically extracted using a constrained Laplacian-based automated segmentation with proximities (CLASP) algorithm.

According to an embodiment, the inner and outer surfaces may have the same number of apices, and a close relationship may be present between the apices corresponding to the inner and outer surfaces. According to an embodiment, a cortical thickness may be defined as a Euclidean distance between the apices connected with the inner and outer surfaces. 40,962 apices may be present in a hemisphere of each brain of an eigen space.

Finally, a thickness value of a cerebral cortex may be computed in a basic brain space rather than a Talairach space due to a limit of linear stereotactic normalization.

An intracranial volume (ICV) may be defined by a total amount of the GM, WM, and CSF, and be calculated by measuring a total volume of voxels in a brain mask. According to an embodiment, the brain mask may be produced using a functional MRI of the brain (FMRIB) software library (FSL) BET (Brain Extraction Tool) algorithm. Since a cortex surface model is produced from an MRI volume transformed into a stereotactic space, an inverse transformation matrix is applied to the cortex surface and the inverse transformation matrix and the cortex surface are reconfigured in a basic space, and thereby the cortical thickness may be measured in the eigen space.

To measure a hippocampus volume (HV), an automated hippocampus division method using a graph cut algorithm coupled with atlas-based division and morphological opening may be used.

### Embodiment 2: Data Analyzing Process

### Embodiment 2-1: Statistical Analysis

According to an embodiment, to compare demographic data and clinical data, a two sample t-test may be used for a continuous variable, and a Chi-square test may be used for a categorical variable. According to an embodiment, all statistical analyses may be performed using R statistical software version 3.5.

### Embodiment 2-2: Tree-Based Model

Whether or not a tau protein load is accumulated may be analyzed using a machine learning algorithm among classification analysis models. According to an embodiment, the machine learning algorithm may be a tree-based model, and the tree-based model enables various analyses of the machine learning algorithm. Further, the tree-based model may naturally process various types of data without pre-processing process. Prediction of multiple trees are aggregated by averaging or majority voting.

According to an embodiment, the tree-based model may be a gradient-boosting machine (GBM) model and a random forest (RF) model (Ridgeway, 2007). Whether or not the tau protein load is accumulated may be analyzed using the GBM model and the RF model, and accuracies of result values of the GBM model and the RF model may be mutually compared. In other words, according to an embodiment, a tau protein accumulation prediction apparatus may perform a machine learning algorithm with the RF model and the GBM model, and classify positivity of the tau protein. The RF model and the GBM model are known as the tree-based model whose performance is continuously excellent. Especially, the GBM model may produce a deriving strong predictions based on weak learners (Friedman, 2000).

### Embodiment 2-3: Validation of Results

Accuracy of the tree-based model may be computed by a 10-fold cross validation (CV) method. K-fold CV may indicate that the data set is divided into unoverlapped K partitions. According to an embodiment, a K-1 data partition is used as a training set that produces a model, and the remaining one set is used as a data set that evaluates a model. This process may be repeated K times.

According to an embodiment, K may be 10. In the case where K=10, accuracy may reach a maximum value. Generalization of predictability and a validation error may be calculated under a CV procedure. When the validation error is a minimum error obtained in the CV procedure, this may be analyzed that a best variable is set.

As an embodiment of a data analyzing process, first, whether or not a tau protein load is accumulated may be analyzed through a machine learning algorithm using clinical information about a subject group as an input. As an embodiment, a tau protein accumulation prediction apparatus (hereinafter referred to as “classifier”) may use only the clinical information as an input.

According to an embodiment, the clinical information may include an age, gender, and educated years of at least one subject, and whether or not MCI is expressed to the subject in an early stage.

According to an embodiment, the classifier using only the clinical information as an input may compare tau protein detectability according to each piece of input information through pieces of additional input information.

According to an embodiment, the classifier may select only at least optimum input information in consideration of expenses compared to the tau protein detectability. The classifier selecting only the optimum input information may discriminate pieces of input information that have a main influence on tau protein detection, and a degree of the influence of the pieces of input information may be estimated by a variable importance score.

According to an embodiment, the input information may be an ADNI data set, and the ADNI data set may include clinical information, neuropsychological test information, brain image information, and gene information about at least one subject.

According to an embodiment, the neuropsychological test information may include at least one of each mini-mental state examination (MMSE), Montreal cognitive assessment (MoCA), and Alzheimer's disease assessment scale-cognitive subscale (ADAS-Cog) test score.

According to an embodiment, the gene information may include ApoE genotype information through a blood test.

According to an embodiment, the brain image information may include 18F-3.0T MRI scanning results, AV45 PET results, and AV1451 PET results.

According to an embodiment, the machine learning model according to the input information of the classifier may include first to seven models.

According to an embodiment, the first to seven models may be divided as in Table 1 according to input information, a variable value, and a structure of the model.

TABLE 1

| Model | Division |
|---|---|
| First | Clinical information (age, gender, and educated years |
| model | of subject), and Stage of MCI(early and late) |
| Second | Clinical information, Stage of MCI(early and late), and |
| model | neuropsychological test information (NP test) |
| Third | Clinical information, Stage of MCI(early and late), NP |
| model | test, and ApoEgene information |
| Fourth | Clinical information, Stage of MCI(early and late), NP |
| model | test, APOE4 genotype information, and PET |
| | information |
| Fifth | Clinical information, Stage of MCI(early and late), NP |
| model | test, APOE4 genotype information, and atrophy |
| | information of hippocampal volume(HV) |
| Sixth | Clinical information, Stage of MCI(early and late), NP |
| model | test, APOE4 genotype information, and atrophy |
| | information of cerebral cortical thickness(Cth) |
| Seventh | Clinical information, Stage of MCI(early and late), NP |
| model | test, APOE4 genotype information, PET information, |
| | atrophy information of hippocampal volume (HV), and |
| | atrophy information of cerebral cortical thickness |
| | (Cth) |

According to an embodiment, the first model is an age, gender, and educated years that are clinical information of a subject, and whether or not MCI is expressed to a subject in an early stage, and a result of whether or not a tau protein is accumulated (positivity or negativity, and hereinafter referred to as “positivity/negativity”) may be predicted. According to an embodiment, the first model may need a medical interview with the subject.

According to an embodiment, the second model may predict positivity/negativity of a tau protein through the constituent factors of the first model and the neuropsychological test information of the subject. The second model may need a medical interview with the subject and a neuropsychological test score.

According to an embodiment, the third model may predict positivity/negativity of a tau protein through the constituent factors of the second model and the APOE4 genotype information of the subject. The third model may need a medical interview with the subject, a neuropsychological test, and an ApoE gene test using blood of the subject.

According to an embodiment, the fourth model may predict positivity/negativity of a tau protein through the constituent factors of the third model, and PET information or FDG PET information of the subject. The fourth model may need a medical interview with the subject, a neuropsychological test, an ApoE gene test using blood of the subject, and an FDG PET test.

According to an embodiment, the fifth model may predict positivity/negativity of a tau protein through the constituent factors of the third model and the atrophy information of the hippocampal volume of the subject. The fifth model may need a medical interview with the subject, a neuropsychological test, an ApoE gene test using blood of the subject, and an MRI test.

According to an embodiment, the sixth model may predict positivity/negativity of a tau protein through the constituent factors of the third model and the atrophy information of the cerebral cortical thickness of the subject. The sixth model may need a medical interview with the subject, a neuropsychological test, an ApoE gene test using blood of the subject, and an MRI test.

According to an embodiment, the seventh model may predict positivity/negativity of a tau protein through the constituent factors of the third model, the PET information or the FDG PET information of the subject, the atrophy information of the hippocampal volume of the subject, and the atrophy information of the cerebral cortical thickness of the subject. The seventh model may need a medical interview with the subject, a neuropsychological test, an ApoE gene test using blood of the subject, a PET test or an FDG PET test, and an MRI test.

According to an embodiment, models that can be configured according to a situation holding input information may be selected from the seven models. The seven models may be analyzed through an RF model and a GBM model among tree-based machine learning algorithms.

According to an embodiment, among the models selected from the seven models, a model showing a best result through an optimization process may be selected as a final model. A positivity/negativity result of the tau protein of the subject may be predicted through the selected model, and importance of factors having an influence on accumulation of the tau protein may be calculated. As an effect of using the tree-based machine learning algorithm, a classification point of time that cannot be found through another machine learning algorithm can be understood.

### Embodiment 2-4: Variable Importance Yardstick

According to an embodiment, the classifier may calculate a variable importance yardstick that measures relative prediction power (prediction intensity) using mean decreased accuracy (MDA) or a GINI according to a model depending on input information. The MDA may indicate a mean decrease in accuracy (MDA) when a relevant variable is replaced with another variable. For example, a variable having an MDA of 15 may be analyzed as a variable that is more important than a variable having an MDA of 5. According to an embodiment, when a variable divides a tree in a tree-based model such as a GBM model and an RF model, a relative importance value of the variable may be calculated by square error loss discordance of all the trees. It can be analyzed that, as the relative importance value becomes higher, an influence of a tau positivity classification variable is great.

### Embodiment 2-5: Partial Dependency

According to an embodiment, a marginal probability density function is measured at each observation point using a method called a partial dependency plot (PDP), and thereby it can be found at which point of time importantly selected input information classifies the positivity/negativity of the tau protein. PDP is a graphic representation tool, and may provide information about whether a specific variable has positive or negative connections with final prediction. For example, in the case where an age is calculated as important input information when the positivity/negativity of the tau protein is classified, an age at a point of time when the positivity/negativity of the tau protein is classified may be measured through PDP analysis. That is, it can be analyzed whether the tau protein is more or less accumulated with age.

According to an embodiment, xₛ may indicate a space of an input variable configured of a selected partial set space, and x_{c} may indicate a parameter space.

xₛ∪x_{c}=x

Then, a functional form of an approximation \hat{ƒ}(x) may be decided depending on two partial set spaces.

\hat{ƒ}(x)=\hat{ƒ}(xₛ,x_{c}), \hat{ƒ}_{c}(xₛ)=\hat{ƒ}(xₛ|x_{c})

If dependence of a complement space is not too strong, a mean function is as follows.

ƒ̅ₛ(xₛ)=Eₓ_{_{c}}[\hat{ƒ}(x)]=∫\hat{ƒ}(xₛ,x_{c})p_{c}(x_{c})dx_{c}

Here, p_{c} (x_{c}) is the marginal probability density function of x_{c}.

Another functional form of the approximation \hat{ƒ}(x) is as follows.

\tilde{ƒ}ₛ(xₛ)=Eₓ[\hat{ƒ}(x)|xₛ]=∫\hat{ƒ}(x)p_{z}(x_{c}|xₛ)dx_{c}

### Embodiment 3: Data Analysis Results

### Embodiment 3-1: Demographic Information and Clinical Characteristics

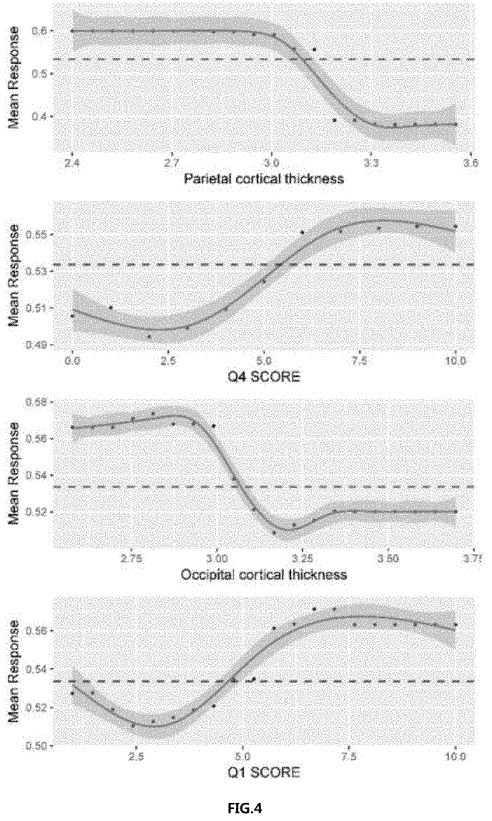

FIG. 4 illustrates demographic information and clinical characteristics.

Referring to FIG. 4, it can be analyzed that there is no significant difference in age (p=0.463), MCI stage (p=0.409), and MMSE (p=0.053) between a negative group of a tau protein and a positive group of a tau protein.

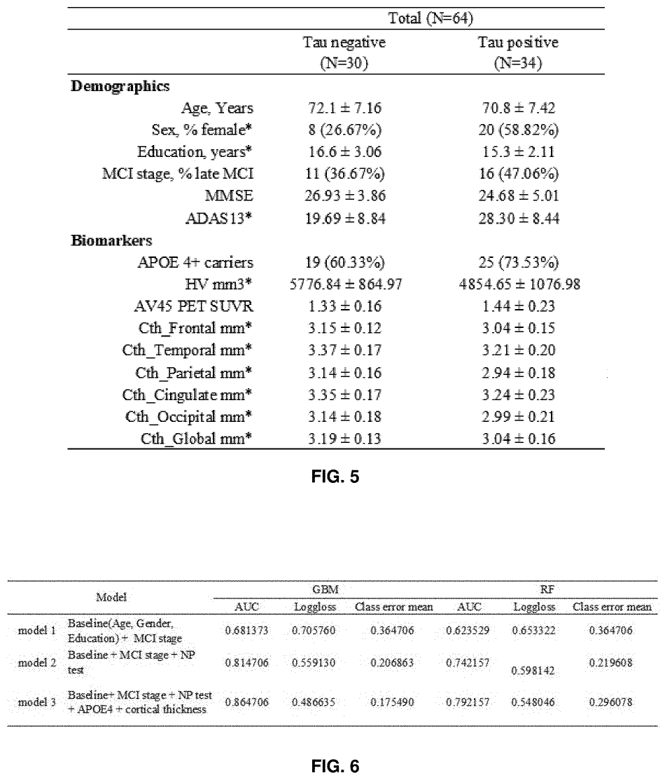

FIG. 5 illustrates a machine learning-based prediction model having a different combination of biomarkers.

Referring to FIG. 5, it can be analyzed that there is no significant difference in ApoE4 carrier and AV45 SUVR in biomarker characteristics of AD but a hippocampal volume (HV) and a cortical thickness in all regions are relatively low in the positive group of the tau protein.

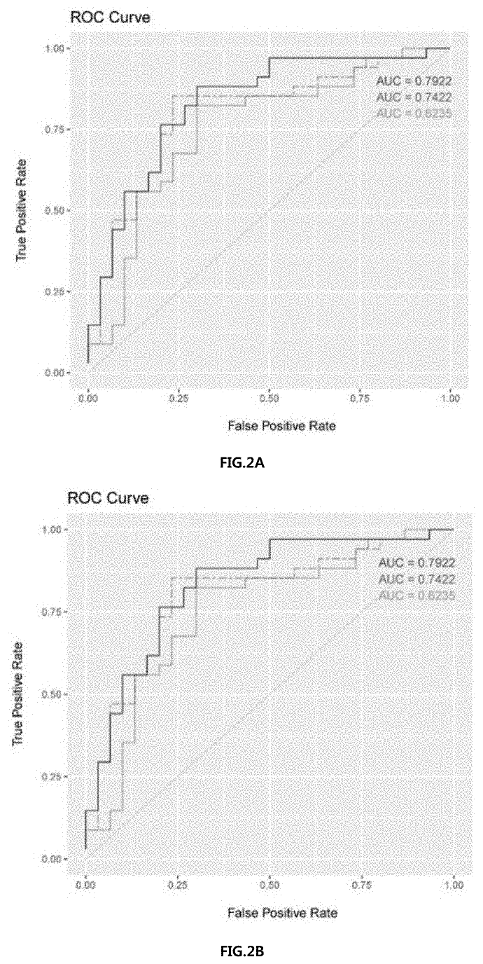

In a machine learning-based prediction model having a different combination of biomarkers, model fitting may be evaluated using graph curves of AUC, loggloss, and class error mean. According to an embodiment, considering that the AUC is high and the loggloss and the class error mean shows a better model about fitting, the seventh model of Table 1 can be analyzed that the data is best fit as in FIGS. 2A and 2B.

### Embodiment 3-2: Relative Function Importance of GBM Model and RF Model

FIG. 6 illustrates relative function importance of a GBM model and an RF model.

Referring to FIG. 6, a characteristic having great relative function importance may indicate greatest contribution to positivity prediction of the tau protein. In a GBM model, a cortical thickness of a parietal lobe may be analyzed as the most important characteristic according to a neuropsychological test of a memory region, a cortical thickness of an occipital lobe, an education age, and an age. The relative function importance of an RF model shows an order similar to the GBM model, the cortical thickness of the parietal lobe, the neuropsychological test of the memory region, the cortical thickness of the occipital lobe, and a hippocampal volume.

According to an embodiment, accuracy of the classifier may be calculated using the area under the receiver operating characteristic (ROC) curve (AUC). The ROC curve is a graph in which a false positive rate (FPR) and a true positive rate (TPR) are placed on x and y axes, respectively. The ROC curve is a curve in which both X and Y are a range of [0,1], and which connects from (0,0) to (1,1). The TPR may indicate sensitivity, and indicate a prediction rate in which data 1 is predicted to be 1. For example, the TPR may indicate a prediction rate in which a cancer patient diagnoses him/her as a cancer. The FPR may indicate a prediction rate in which data 0 is falsely predicted to be 1. For example, the FPR may indicate a prediction rate in which a patient other than a cancer patient is diagnosed as a cancer. The AUC of the ROC curve may indicate a value by which a base area of the ROC curve is obtained. It can be analyzed that, as the AUC of the ROC curve becomes closer to 1, prediction performance is good.

According to an embodiment, the classifier may predict positivity of a tau protein of a prodromal AD subject in which the AUC of the GBM model has 0.865 and the AUC of the RF model has 0.792.

According to an embodiment, in the classifier using only clinical information (age, gender, and educated years) of a subject as input information, the AUC in the GBM model is increased from 0.681 to 0.815 when information about a neuropsychological test is added, and the AUC in the GBM model is further increased to 0.865 when brain MRI information is added.

FIG. 2A illustrates an ROC curve of a GBM model, and FIG. 2B illustrates an ROC curve of an RF model.

Referring to FIGS. 2A and 2B, it can be analyzed that classifier model 3 based on GBM has highest accuracy.

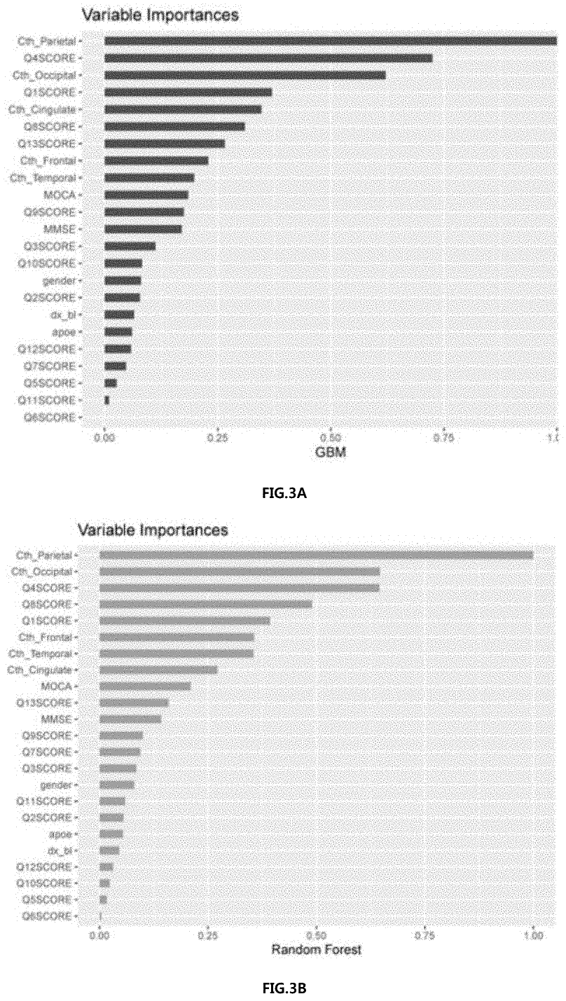

FIG. 3A illustrates importance of factors having an influence on accumulation of a tau protein in a GBM model, and FIG. 3B illustrates importance of factors having an influence on accumulation of a tau protein in an RF model.

Referring to FIGS. 3A and 3B, it can be analyzed that variables having a greatest influence on discriminating positivity and negativity of a tau protein in both of the GBM model and the RF model are a cortical thickness of a parietal lobe and a memory item of a cognitive function test.

Further, it can be analyzed that a cortical thickness of a parietal lobe, an occipital lobe, a neuropsychological test of a memory region, and a gender are input information having a more main influence through order analysis of variables having an influence on a tau protein accumulation prediction apparatus.

While the present disclosure has been described above in detail through the exemplary embodiments, it will be understood by those having ordinary knowledge in the art to which the present disclosure pertains that the above-described embodiments can be variously modified without departing from the category of the present disclosure. Therefore, the right scope of the present disclosure should not be limited to the above-described embodiments, and should be defined by not only the following claims but also all changed or modified forms derived from the concepts equivalent to the claims.

What is claimed is:

1. A tau protein accumulation prediction method comprising:
a process of inputting: at least one of neuropsychological test information, APOE4 genotype information, positron emission tomography (PET) information, atrophy information of a hippocampal volume, and atrophy information of a cerebral cortical thickness; clinical information; and mild cognitive impairment expression stage information; and
a process of calculating a prediction result indicating whether or not a tau protein is accumulated on the brain.

2. The tau protein accumulation prediction method of claim 1, wherein the process of calculating a prediction result includes a process of analyzing whether or not a tau protein load is accumulated, using a machine learning algorithm out of classification analysis models.

3. The tau protein accumulation prediction method of claim 2, wherein the machine learning algorithm includes a tree-based model.

4. The tau protein accumulation prediction method of claim 3, wherein the tree-based model includes one of a gradient boosting machine (GBM) model and a random forest (RF) model.

5. The tau protein accumulation prediction method of claim 1, wherein the clinical information includes at least one of an age, a gender, and educated years of a subject.

6. A tau protein accumulation prediction apparatus comprising:
an input unit configured to receive: at least one of neuropsychological test information, APOE4 genotype information, positron emission tomography (PET) information, atrophy information of a hippocampal volume, and atrophy information of a cerebral cortical thickness; clinical information; and mild cognitive impairment expression stage information; and
a processor configured to calculate a prediction result indicating whether or not a tau protein is accumulated on the brain.

7. The tau protein accumulation prediction apparatus of claim 6, wherein the processor analyzes whether or not a tau protein load is accumulated, using a machine learning algorithm out of classification analysis models.

8. The tau protein accumulation prediction apparatus of claim 7, wherein the machine learning algorithm includes a tree-based model.

9. The tau protein accumulation prediction apparatus of claim 8, wherein the tree-based model includes one of a gradient boosting machine (GBM) model and a random forest (RF) model.

10. The tau protein accumulation prediction apparatus of claim 6, wherein the clinical information includes at least one of an age, a gender, and educated years of a subject.
